(19) United States
(12) Patent Application Publication
(10) Pub. No.: US 2022/0331213 A1
(43) Pub. Date: Oct. 20, 2022

(54) ANTIMICROBIAL COMPOSITIONS COMPRISING A BENZOIC ACID ANALOG AND ACID PH BUFFERS

(71) Applicant: eHealth Nexus LLC, Phoenix, AZ (US)

(72) Inventor: Michael Joseph Mobley, Phoenix, AZ (US)

(73) Assignee: eHealth Nexus LLC, Phoenix, AZ (US)

(21) Appl. No.: 17/723,335

(22) Filed: Apr. 18, 2022

Related U.S. Application Data
(60) Provisional application No. 63/176,136, filed on Apr. 16, 2021.

(51) Int. Cl. A61K 8/368; A01N 59/20; A01N 25/02; A01N 37/36; A01N 37/04; A01N 37/02; A61K 8/19; A61K 8/362; A61K 8/36; A61K 8/49; A01P 1/00; A61Q 17/00

(52) U.S. Cl. CPC A61K 8/368; A01N 59/20; A01N 25/02; A01N 37/36; A01N 37/04; A01N 37/02; A61K 8/19; A61K 8/362; A61K 8/36; A61K 8/4926; A01P 1/00; A61Q 17/005

(57) ABSTRACT

Various embodiments provide an antimicrobial composition comprising: a safe and effective amount of benzoic acid analog or analogs; a safe and effective amount of a buffer system of weak organic acids buffer with the negative log of the acid dissociation constant, pKa, between about 3.2 and 4.9; and a dermatologically acceptable carrier for the benzoic acid analogs and weak organic acid buffers, wherein the antimicrobial composition has a pH from about 3.2 to 4.9. In some embodiments, the antimicrobial composition comprises a safe and effective amount of an antimicrobial metal ion salt, a preferred salt being copper gluconate, at a level and pH that does not precipitate with the weak acid buffers.

### RELATED PATENT APPLICATIONS

This patent application claims priority to and the benefit of U.S. Provisional Patent Application Ser. No. 63/176,136 filed Apr. 16, 2021, which is incorporated by reference herein.

### BACKGROUND

Compositions with benzoic acid and several analogs of benzoic acid, such as salicylic acid, have both immediate, as well as residual antimicrobial activity when used on the skin. These compositions may have antibacterial, antifungal, and antiviral activity. These compositions are useful in products such as antiseptic hand sanitizers, skin wipes, skin sprays, salves, and ointments. A broad range for the pH values have been reported for these compositions, and some evidence indicates that an acidic pH is preferred for the efficacy against certain pathogens. The lower pH may be useful in the denaturing of proteins and membranes on a bacterium or in disrupting the lipid envelope or capsid layer of many viruses.

The natural pH of the skin is acidic, which contributes to the antimicrobial defense of the skin toward certain germs. Various studies of the pH of the skin, have reported an apparent pH gradient with the outermost layer of the skin nearer to neutrality having a pH of about 5.5 or higher and a lower level, the “acid mantle” of the skin, having a pH of about 4.5-5.0, which is buffered with natural fatty acids produced in the skin. Of course, there are variations in this pH profile of the skin based upon the location of the skin. In addition, the pH of the skin may also vary depending on exposure to materials placed upon the skin such as alkaline soaps, moisturizers, sunscreens, and insect repellents. For example, frequent hand washing with soap can elevate the pH of the surface skin of the hands.

When the compositions containing analogs of benzoic acid in the acidic pH range are applied to surfaces, these compositions can cause significant reductions in the viability of many bacteria and viruses. However, the residual antimicrobial activity of these compositions applied to skin diminishes with time. For example, the residual anti-rhinovirus activity of 2% salicylic acid on the skin was shown to diminish over 3 hours. It is suggested here this is related to the increasing pH of the skin surface with time as the skin is buffered and the outer surface of the skin gradually returns to its natural level of about pH 5.5 or above, which lowers the effectiveness of these compositions.

### SUMMARY

Various embodiments provide an antimicrobial composition comprising: an effective amount of one or more benzoic acid analogs in a weight percent between 0.1% and 20% of the composition; a combination of weak organic acid buffers, having at least 3 different pKa values between 3.2 and 4.9, in a combined weight present between 0.15%-7% of the composition; an effective amount of an antimicrobial copper ion salt in a weight percent of copper of between 0.0025%-0.2% of the composition; and an amount of glycine in 1.5 to 4 times the molar concentration of copper ions in the formulation.

In some embodiments, the one or more benzoic acid analogs can be selected from the group consisting of salicylic acid, benzoic acid, salts thereof, and combinations thereof. In some embodiments, the antimicrobial copper ion salt can be copper gluconate. In some embodiments, the combination of weak acid buffers can be a combination of adipic acid (pKa=4.43), nicotinic acid (pKa=4.82), and acetic acid (pKa=4.76).

Various embodiments provide an antimicrobial composition comprising: a safe and effective amount of benzoic acid analog or analogs; a safe and effective amount of a buffer system of weak organic acids buffer with the negative log of the acid dissociation constant, pKa, between about 3.2 and 4.9; and a dermatologically acceptable carrier for the benzoic acid analogs and weak organic acid buffers, wherein the antimicrobial composition has a pH from about 3.2 to 4.9.

In some embodiments, the antimicrobial composition comprises a safe and effective amount of an antimicrobial metal ion salt, a preferred salt being copper gluconate, at a level and pH that does not precipitate with the weak acid buffers.

In some embodiments, the copper ions are complexed with a coordinating or chelating agent that lowers the free copper ion concentration, a preferred agent being glycine, thereby preventing the precipitation of the copper ion with the weak acid buffers.

These embodiments, as well as, other embodiments, additional features, certain configurations, and exemplary applications, are described in the following drawings and description.

### DRAWINGS

The present disclosure will become more fully understood from the description and the accompanying drawings, which are published in color, wherein:

FIG. 1 is a graph illustrating the dissociation of salicylic acid;

FIG. 2 is a graph illustrating the dissociation of several weak organic acids;

FIG. 3 is a graph illustrating the base (NaOH) titration of an exemplary formulation of compared to a reference formulation, which quantitates the increased pH buffering from an exemplary combination of weak acids, according to various embodiments;

FIG. 4 is an exemplary photo illustrating a comparison of an exemplary formulation of copper complexation with glycine addition (dark blue) to an example of a formulation of copper gluconate (light green), according to various embodiments;

FIG. 5 is an exemplary photo illustrating a comparison of a known hand sanitizer formulation containing salicylic acid and a copper salt, aged 14 months (brown), to an exemplary formulation containing a copper complexing agent aged 14 months (blue), according to various embodiments;

FIG. 6 is an exemplary photo illustrating a comparison of the known hand sanitizer formulation containing salicylic acid and a copper salt, freshly prepared, (light green), to the known hand sanitizer formulation containing salicylic acid and a copper salt, aged 14 months (brown), according to various embodiments; and

FIG. 7 is an exemplary photo illustrating a comparison of the known hand sanitizer formulation containing salicylic acid and a copper salt, copper chloride dihydrate (light green on left), compared with the same formulation with glycine added to complex the copper (blue on right);

The drawings described herein are for illustrative purposes only of selected embodiments and are not intended to represent all possible implementations and are not intended to limit the scope of any of the exemplary embodiments disclosed herein or any equivalents thereof.

The patent or application file contains at least one drawing executed in color. Copies of this patent or patent application publication with color drawing(s) will be provided by the Office upon request and payment of the necessary fee.

### DESCRIPTION

The following description is merely exemplary in nature and is in no way intended to limit the exemplary embodiments, their applications, or uses. It is understood that the steps within a method may be executed in different order without altering the principles of the present disclosure. For example, various embodiments may be described herein in terms of various functional components and processing steps. It should be appreciated that such components and steps may be realized by any number of hardware components configured to perform the specified functions.

Various embodiments provide antimicrobial compositions comprising one or more benzoic acid analogs and one or more weak organic acid buffer having a pKa between 3.2 and 4.9. In some embodiments, the antimicrobial compositions can further comprise a dermatologically acceptable carrier. In some embodiments, the antimicrobial compositions can further comprise an alcohol or a combination of alcohols. In some embodiments, the antimicrobial compositions can further comprise a copper ionic salt, which disassociates in water and/or water-alcohol solutions.

As is known in the art, an antimicrobial composition kills or inhibits the growth of microorganisms but causes little or no damage to the host. Antimicrobial compositions have one or more effect of an antibacterial, an antiviral, an antifungal, and an antiprotozoal. The antimicrobial compositions of the present invention are efficacious for rapidly cleaning surfaces which are infected or contaminated with Gram negative bacteria, Gram positive bacteria, viruses (e.g., rhinoviruses, adenoviruses, rotaviruses, herpes viruses, respiratory syncytial viruses, coronaviruses, parainfluenza viruses, enteroviruses, influenza viruses, etc.), and the like and are also able to provide residual anti-viral and antibacterial effectiveness against such microorganisms. These antimicrobial compositions are highly efficacious for applications to the skin, for example as a hand sanitizer or a sanitizing wipe. In addition, these antimicrobial compositions can be highly efficacious for personal care applications, household cleaning applications, and industrial and hospital applications.

In an acidic environment having a low pH, the antimicrobial compositions containing analogs of benzoic acid cause significant reductions in the viability of many bacteria and viruses. However, when these antimicrobial compositions, in the acidic environment, are applied to a skin surface, the residual antimicrobial activity diminishes with time, which is due to the fact the natural pH of the skin increases the pH of the acidic environment. Over time, the increasing pH of the skin surface gradually returns to its natural level of about pH 5.5 or above, which lowers the effectiveness of these compositions.

Based on unexpected results, the present invention solves this problem by increasing the potency and duration of the antimicrobial compositions containing benzoic acid analogs on the skin through the addition of weak acid compounds with acid dissociation constants, pKa values, between 3.2 and 4.9 as these will retain the lower pH of the skin for a longer time.

To offset the skin's natural tendency ability to move buffer toward a higher pH of between 4.5 and 5.5, the concentration of benzoic acid analog in an antimicrobial composition for skin application could be increased as such weak organic acids will have some tendency to increase the buffering capacity of the composition. For example, benzoic acid has an acid dissociation constant, pKa, of about 4.2 and thus a formulation with a pH below about 4.5 will have a capacity to buffer the surface of the skin at a pH of 4.5 and below. Increasing benzoic acid concentration would increase the time period when the pH of the skin is held to below 5.0, thereby potentially imparting improved efficacy, however, the increased benzoic acid concentration may cause irritation and/or damage the skin surface.

In addition, increasing the concentration of benzoic acid and its analogs will have its issues as these are pharmacologically active compounds that can have concerns regarding irritation, sensitivity, or toxicity at higher concentrations. Thus, it is desirable to increase the duration of antimicrobial activity of these benzoic acid analogs, without increasing their concentrations.

Accordingly, various embodiments, as described herein, provide an antimicrobial formulation comprising: an effective amount of one or more benzoic acid analogs; a combination of weak organic acid buffers having at least 3 different pKa values between 3.2 and 4.9; an effective amount of an antimicrobial copper ion salt; and glycine.

Benzoic Acid and Benzoic Acid Analogs.

The antimicrobial compositions of the present invention comprise a safe and effective amount of a benzoic acid analog. Benzoic acid analogs include those having the structure:

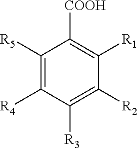

Wherein R1, R2, R3, R4, and R5, represent a variety of functional groups which are substituted independently, as known by those of skill in the art. Any benzoic acid analog or combinations thereof can be used in the antimicrobial compositions of the present disclosure.

In some embodiments, a benzoic acid analog is selected from the group consisting of salicylic acid, benzoic acid, and combinations thereof. In some embodiments, a benzoic acid analog can be a salt of a benzoic analog, for example sodium salicylate, copper salicylate, or sodium benzoate. In some embodiments, a benzoic acid analog can be a combination of a salt of a benzoic analog, and at least one of salicylic acid and benzoic acid. In some embodiments, a benzoic acid analog is selected from the group consisting of salicylic acid, benzoic acid, salts thereof, and combinations thereof.

In some embodiments directed to COVID-19, the benzoic acid analog can be nafamostat, camostat, and/or bromhexine, which inhibits cellular transmembrane serine protease 2 (TMPRSS2) both early and late endosome entry processes. Accordingly, one or more of such benzoic analogs can effectively inhibit the activity of COVID-19 on a skin surface. In some embodiments, one or more of such benzoic analogs can effectively kill COVID-19 on a skin surface. Methods herein include preventing a spread of COVID-19 by treating a skin surface with an antimicrobial composition, as described herein, wherein the benzoic acid analog is at least one of nafamostat, camostat, and bromhexine.

The compositions of the present disclosure can comprise from about 0.1% to about 20%, of the benzoic acid analog, by weight of the composition. In some embodiments, the compositions can comprise from about 0.1% to about 10% of the benzoic acid analog(s), by weight of the composition. In some embodiments, the compositions can comprise from about 0.25% to about 5% of the benzoic acid analog(s), by weight of the composition. In some embodiments, the compositions can comprise from about 1% to about 4% of the benzoic acid analog(s), by weight of the composition.

Weak Acid Buffer.

In accordance with the unexpected results of the invention, various novel antimicrobial compositions, described herein, increase the potency and duration of antimicrobial compositions containing benzoic acid analogs on the skin through the addition of weak acid compounds with acid dissociation constants (pKa values) in a range of between 3.2 and 4.9, which have been shown to retain a lower pH of the skin for a longer time period.

Various weak acids, which are included in the antimicrobial compositions, as described herein, are all generally recognized as safe (“GRAS”) for skin formulations, as such, do not contribute to the toxicity, the sensitizing, or the irritation of the formulations of the antimicrobial compositions. The aqueous pKa values for many common organic acids are well known to those of skill in the art. There are many such weak acids that are generally recognized as safe at the lower concentrations. Some examples of weak acids useful herein include: gluconic acid (pKa˜3.85), lactic acid (pKa˜3.8), glycolic acid (pKa˜3.8), acetic acid (pKa˜4.8). The buffering of the antimicrobial compositions by weak acids can be achieved through the addition of the weak acid, or a combination of the weak acid with a salt of the weak acid, or a combination of the salt of the weak acid with a stronger acid. For example, buffering from gluconic acid can be achieved by addition of gluconic acid, addition of gluconic acid with sodium gluconate, or the addition of sodium gluconate with salicylic acid. Therefore, the salts of weak acids in the antimicrobial compositions are included by reference in this disclosure of suitable weak acids.

Such weak acids may be selected from the group of naturally occurring fatty acids in the skin, which have dissociation constants in range of between 3.2 and 4.9. Examples of such fatty acids are stearic acid or palmitic acid that have pKa values between 4.5 and 5.0. Such fatty acid pH buffers can have an additional function in the formulation, serving as a surfactant or emollient. Due to this surface-active activity, such fatty acids may also contribute antimicrobial activity to the antimicrobial compositions.

Other preferred organic acids include the group of dicarboxylic acids, where the dissociation constants for one or both acid dissociations are in the range of between 3.2 and 4.9. For example, adipic acid (hexanedioic acid) has an aqueous pK1 of about 4.4 and a pK2 of about 5.4. Similarly, succinic acid (butanedioic acid) has a pK1 of about 4.2 and a pK2 of about 5.6. Glutaric acid (pentanedioic acid) has a pK1 of about 4.3 and a pK2 of about 5.4. The dicarboxylic acids are useful as they can provide twice the buffering ability per molar concentration as the monocarboxylic acids.

Other preferred acids are the polycarboxylic acids with 3 or more carboxylic acid groups on the molecule with dissociation constants in the range of between 3.2 and 4.9. This would include, for example, citric acid, a tricarboxylic acid. The pK1 for citric acid is outside the preferred range at about 2.9. However, the pK2 for citric acid is in the range of 4.5 to 5.0 making it a useful buffer. This group would include the polymeric acids. An example of a polymeric acid is polyacrylic acid that is a polymer of varying lengths with a dissociation constant, pKa, of about 4.5 for the carboxylate groups. Similar polymers, copolymers, and cross-linked polymers are anticipated with the requirement that they can pH buffer in the preferred range and have acid dissociation constants in the preferred range. Such pH buffers can have an additional function in a formulation as a thickening agent or a suspending agent, however, these additional functions are not required in the formulations of the antimicrobial compositions.

Another preferred grouping of weak acid buffers are nutritional acids. Such nutritional acids include derivatives of vitamins such as nicotinic acid (niacin) with a pKa˜4.75 and ascorbic acid (Vitamin C), pKa˜4.2.

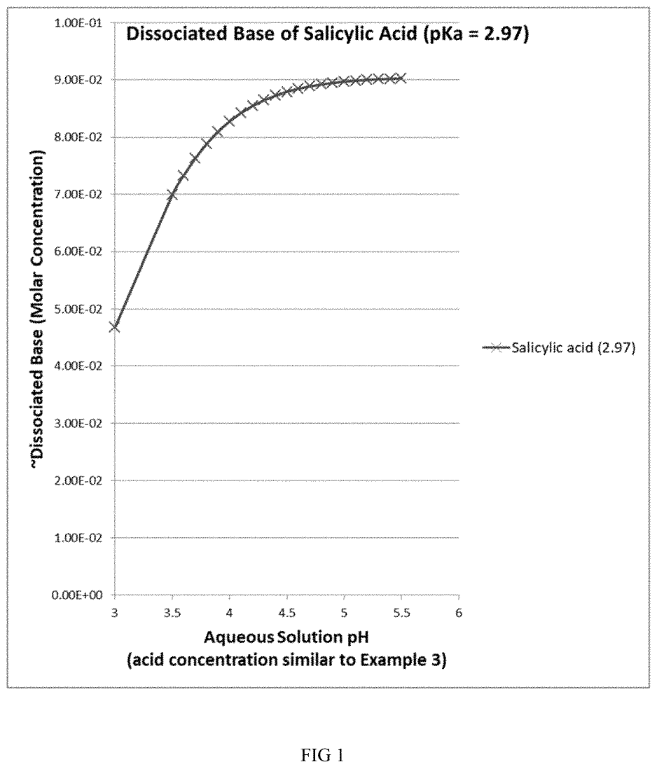

As an example of how the present invention can be applied, a composition of 2% salicylic acid at a pH of 4.2 will have very little buffering capacity and the pH can be raised quickly on the skin. As illustrated in FIG. 1, the dissociation constant of salicylic acid, the pKa, is about 3. Thus, there is very little associated salicylic acid and little buffering capacity for the formula placed on the skin. The formula pH of 4.2 can be a desirable pH for formula composition flexibility, thickening, and skin pH compatibility. The buffering capacity can be greatly increased by the addition of weak acids, for example, adipic acid at 0.6% which has a pK1 of 4.4. This will significantly increase the buffering of a composition at pH 4.2 and retain the antimicrobial activity of the salicylic acid much longer. Alternatively, this can allow the reduction in the total concentration of salicylic acid, say to 1%, without a reduction in the overall antimicrobial activity.

The weak acid buffers will be effective in adding to the total buffering of the formulation placed on the skin if the concentration of associated acid in the formula is sufficiently high at the pH of the formula and significantly exceeds the buffering of the formula with the benzoic acid analog alone. The concentration of associated weak acids can be determined through a base (e.g., NaOH solution) titration of the formula up to a pH of 5.7. The effective concentration is dependent on the type of product, e.g., hand sanitizer, wipe, spray, or ointment, and the amount of product delivered per square centimeter of skin. In a hand sanitizer, the effective concentration range for total associated acid buffers is between about 3×10⁻² and 2×10⁻¹ molal (or molar) equivalents. This buffering by weak acid buffers added should exceed the buffering of the benzoic acid analogs only formula by about 2×10⁻² molal (molar). When delivered in another product form such as a sanitizer wipe, spray, or ointment, the concentration of titratable weak acids buffering should be in the range from 5×10⁻⁷ to 10⁻⁵ moles (or OH⁻ titration equivalents) per square centimeter applied to the skin surface.

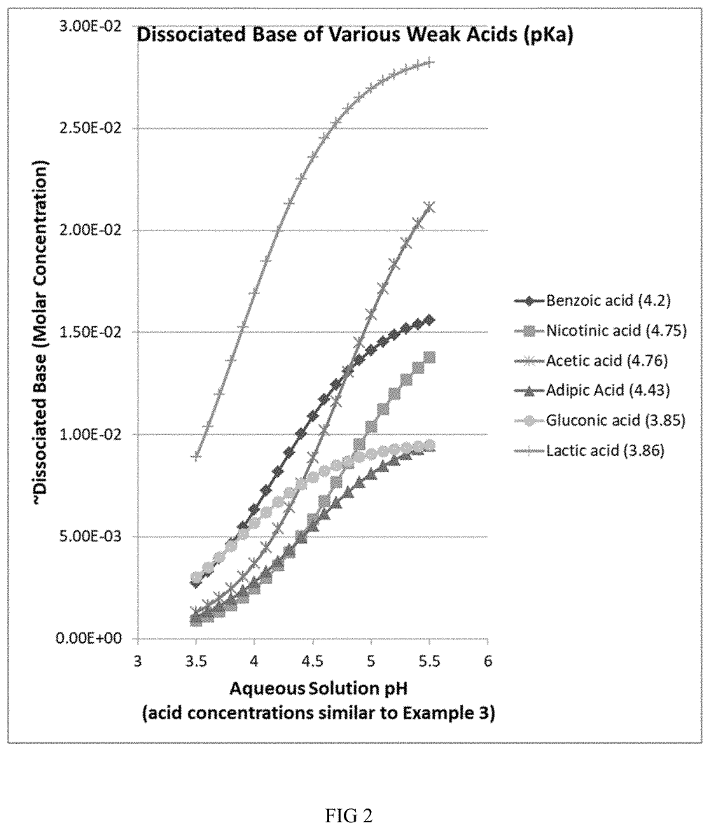

With reference to FIG. 2, a graph, similar to FIG. 1 but with a different concentration scale, illustrates examples of theoretical calculations of various un-associated weak acids by pH. The calculation uses approximate concentrations of acid in an aqueous formula based upon the pKa values of the acids. The graph includes calculations for benzoic acid, nicotinic acid, acetic acid, adipic acid, gluconic acid, lactic acid. The graph shows most of the buffering capacity for these selected weak acids at a pH of about 3.6.

For example, at pH of 3.6, most acids are associated with the exception of salicylic acid. This provides stability of the formulation, without going too low in pH. These general characteristics of weak acids can translate to hydro-alcoholic solutions and formulations where the precise pKa values will change, but his trend of lowering the concentration of unassociated base for the weak acids in this pH range will still hold. In another exemplary formulation, nicotinic acid removed from the previous exemplary formulation and replaced with niacinamide, which gradually hydrolyses to nicotinic acid below pH of 4.0.

In some embodiments, a buffering system of multiple weak acids in low concentrations can be a combination of weak organic buffers, all with a pKa value in a range between about 3.2 and about 4.9. In some embodiments, the buffering system of multiple weak acids in low concentrations can be a combination of three of weak organic buffers, all with a different pKa values between about 3.7 and about 4.9. In unexpected results, the buffering system of multiple weak acids in low concentrations in this pKa range is more effective in preventing the precipitation of the copper ion as compared to the use of only one weak acid buffer.

In some examples, the buffering system of multiple weak acids in low concentrations can be a combination of adipic acid (pKa=4.43), nicotinic acid (pKa=4.82), and acetic acid (pKa=4.76). In some examples, the buffering system of multiple weak acids in low concentrations can be a combination of three or more of: adipic acid (pKa=4.43), nicotinic acid (pKa=4.82), glutaric acid (pKa=3.76), gluconic acid (pKa=3.85), lactic acid (pKa=3.86), and acetic acid (pKa=4.76).

As illustrated in FIG. 2, a base titration of an exemplary formulation of an antimicrobial composition can determine the buffering capacity of various weak acids. The results illustrated in FIG. 2 plots the titration of a hand sanitizer prepared with exemplary formulation described in Example 3. The formulation is diluted 3:1 with water to measure the initial pH and to titrate with 0.10 N Sodium Hydroxide water solution, plotting the pH for the milliequivalents of OH⁻ added per gram of sanitizer.

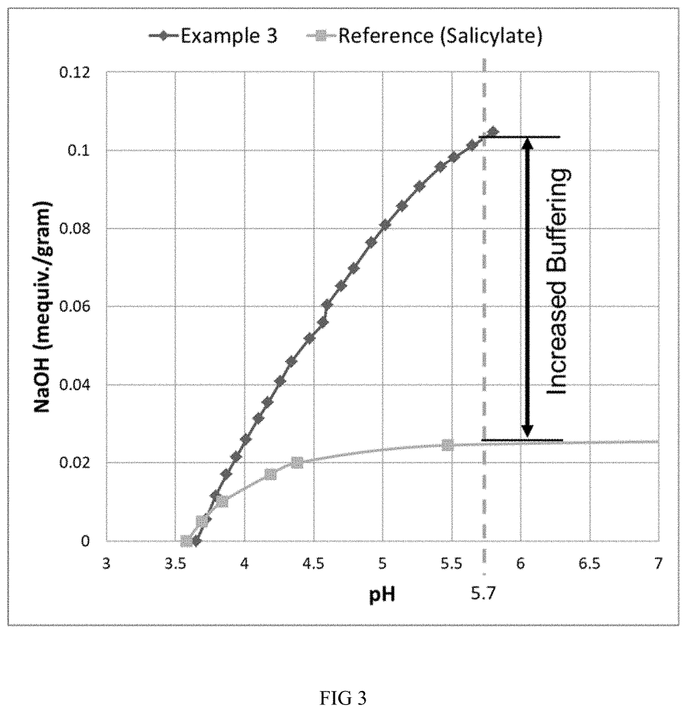

Illustrated in FIG. 3, a comparison in the titration of a Reference formulation (Salicylate with similar levels of salicylic acid and salicylate to Example 3 without the combination of weak acids, which provide the buffering) to the titration of with the exemplary formulation described in Example 3. The base required for the titration of each formula can be compared up to the pH of 5.7. The Reference formulation, which required 0.025 mequiv./gram (or 0.025 molal) is compared to the exemplary formulation described in Example 3, which required 0.102 mequiv./gram (or 0.102 molal) for the titration. This result illustrates the increased buffering capacity of the exemplary formulation described in Example 3 of 0.077 mequiv./gram (0.077 molal) as compared to the Reference. This demonstrates that the concentration for effective buffering of the exemplary formulation described in Example 3 (0.102 molal) is within the range for this invention of between about 3×10⁻² and 2×10⁻¹ molal (or molar) equivalents. In addition, this demonstrates the buffering of the exemplary formulation described in Example 3 significantly exceeds the buffering of the Reference A (Salicylate) formulation.

The formulation for this Reference A (Table 1) titration comparison is:

TABLE 1

| Ingredient (Wt. %) | Reference A |
|---|---|
| Ethanol (SD alcohol 40-B, 200 | 60.00% |
| proof) (v/v) | |
| Salicylic acid | 0.35% |
| Sodium salicylate | 1.00% |
| Propylene Glycol | 1.00% |
| Glycerin | 3.60% |
| DL Panthenol | 0.80% |
| Hydroxypropyl cellulose | 0.55% |
| (Klucel HF) | |
| Sodium lauryl sulfate | 0.60% |
| pH (Adjust to pH | |
| HCl or 10 N NaOH) | |
| Water, USP Q.S to 100% | |

The exemplary formulation described in Example 3 (Table 2) for titration comparison is:

TABLE 2

| Ingredient (Wt. %) | Example 3 |
|---|---|
| Ethanol (SD alcohol 40-B, 200 | 60.00% |
| proof) (v/v) | |
| Salicylic acid | 0.35% |
| Benzoic acid | 0.20% |
| Sodium salicylate | 1.00% |
| Copper gluconate | 0.22% |
| Glycine | 0.15% |
| Lactic acid | 0.26% |
| Adipic Acid | 0.15% |
| Glacial Acetic Acid | 0.12% |
| Propylene Glycol | 1.00% |
| Glycerin | 3.60% |
| Niacinamide | 0.50% |
| DL Panthenol | 0.80% |
| Hydroxypropyl cellulose | 0.55% |
| (Klucel HF) | |
| Sodium lauryl sulfate | 0.60% |
| pH (Adjust to pH 3.6 with 6 N | |
| HCl or 10 N NaOH) | |
| Water, USP Q.S to 100% | |

As illustrated in the Tables 1 and 2, both formulations have the same weight percentage of ethanol (60%), salicylic acid (0.35%), and sodium salicylate (1.00%). In contrast to Reference A, the exemplary formulation described in Example 3 includes a buffering system of multiple weak acids in low concentrations: lactic acid (pKa=3.86, 0.26%), adipic acid (pKa=4.43, 0.15%), and glacial acetic acid (pKa=4.76, 0.12%). In addition, the exemplary formulation described in Example 3 includes benzoic acid (pKa=4.2, 0.20%), which can increase the buffering capacity of the formulation and/or increase the antimicrobial activity of the formulation.

In some embodiments, the desired buffering capacity can be achieved by adding components that are not weak acid buffers, but that react in the formulation or on application of the formulation to the skin surface to form a weak acid buffer. An example component is acetic acid ethyl ester. In acidic conditions, acetic acid ethyl ester hydrolyzes to acetic acid and ethanol. Another example component is niacinamide, which will hydrolyze to nicotinic acid.

Metal Ions and Copper Salts

The patent literature teaches that the antimicrobial activity of benzoic acid analogs can be combined with metal salts. See for example: U.S. Pat. No. 6,294,186, entitled ANTIMICROBIAL COMPOSITIONS COMPRISING A BENZOIC ACID ANALOG AND A METAL SALT, which includes inventors Peter William Beerse, Kimberly Ann Biedermann, Steven Hardy Page, Michael Joseph Mobley, and Jeffrey Michael Morgan, which is incorporated by reference. Many metals have been described with desirable properties. Preferred metals ions can include Mn, Ag, Zn, Sn, Fe, Cu, Al, Ni, Co, Ti, Zr, Cr, La, and combinations thereof. However, more preferred metal ions can disassociate from salts of metals selected from the group consisting of Cu, Fe, Sn, Zn, Ag, Ni, and combinations thereof.

However, the addition of soluble metal salts to compositions that contain weak acid buffers can result in unstable formulations with the precipitation of the metal ion as a metal salt of the unassociated base A− (where HA is the associated weak acid). In unexpected results, the inventor solved this problem using a buffering system of multiple weak acids in low concentrations, ensuring that the concentration of each weak acid remains below the solubility product of the metal-base salt. This is further achieved by lowering the composition pH to about a half pH unit or more below the pKa of the weak acid, thus reducing the concentration of unassociated base, [A-] in the composition—thereby reducing the potential for precipitation of the metal-base salt.

Preferred embodiments, described herein, include soluble copper salts. In some embodiments, the copper salt is a metal salt or complex of acetates, ascorbates, chlorides, benzoates, citrates, fumarates, gluconates, glutarates, lactates, malates, malonates, salicylates, succinates, sulfates, undecylates, and combinations thereof.

An example of a preferred copper salt is copper gluconate, which is the copper salt of gluconic acid. It is an odorless light blue or blue-green crystal or powder which is easily soluble in water and is insoluble in ethanol.

Copper has potent biocidal properties. Copper gluconate is listed as a dietary supplement to provide copper to the body. The typical dose is 2.0 mg copper per day. Copper can be involved in numerous physiological and metabolic processes critical for the appropriate functioning of almost all tissues in the human body. In the skin, copper is involved in the synthesis and stabilization of extracellular matrix skin proteins and angiogenesis.

Copper gluconate repairs skin from within, meaning it firms the skin by improving collagen and elastin. Visible signs of aging like a fine line or wrinkles can be noticeably improved because of regulation of different skin cell related processes. It revitalizes skin by providing elemental or ionic copper. It also acts as an anti-inflammatory and helps modulate the inflammation process and decreases the allergic response of skin to external allergens. This process leads to natural and calm skin.

It is desirable to have the soluble copper level in a range that provides desirable activity without being at a high level that can lead to toxicity. A preferred range for a skin sanitizer can be in a range between 25 ppm and 2000 ppm soluble cooper, with a more preferred range between 100 ppm and 1200 ppm copper.

When a copper salt is added to the formulation, there is the potential for the copper to precipitate with the weak acid base. Unexpected findings resulted in an ideal buffering system of multiple weak acids being a combination of several weak acids that keeps the concentration of each low. Using a combination of suitable weak acids keeps the level of a single free base below the level that might cause precipitation of the copper salt. Using a combination of weak acids and a lower pH to suppress the level of free acid can be used to prevent the precipitation of copper salts.

When one or more benzoic analogs are included in formulations having a copper salt, the benzoic analog complexes with the Cu+2 ion and the Cu+2 complex is insoluble and thus the complex precipitates out of the solution. Increasing the concentration of the one or more benzoic analogs may lower a pH of a formulation, however, the increase of the concentration of the one or more benzoic analogs will increase the concentration of the insoluble complex

An additional means of reducing the potential for copper precipitation with the weak acids, thereby allowing increased total concentrations of the acid and the soluble copper, is to add an agent to form a soluble complex of the copper +2 ion. Such complexes can reduce the availability of Cu+2 free ions to react with the weak acids and the benzoic acid derivatives and thus reduce the potential for precipitation or instability of the formula. An ideal Cu+2 complexing agent found was glycine which forms a coordination complex around Cu+2 when the glycine concentration exceeds the Cu concentration by more than 2:1. The copper glycine complex is highly soluble. Glycine, as a natural amino acid, is also very safe. In addition, the copper glycine complex lowers the concentration of free Cu+2 ions in a formulation, which lowers a potential for the formulation to oxidize. The oxidation of the free Cu+2 ions in a formulation degrades the formulation and over time, can turn the color of the formulation to a rusty brown color.

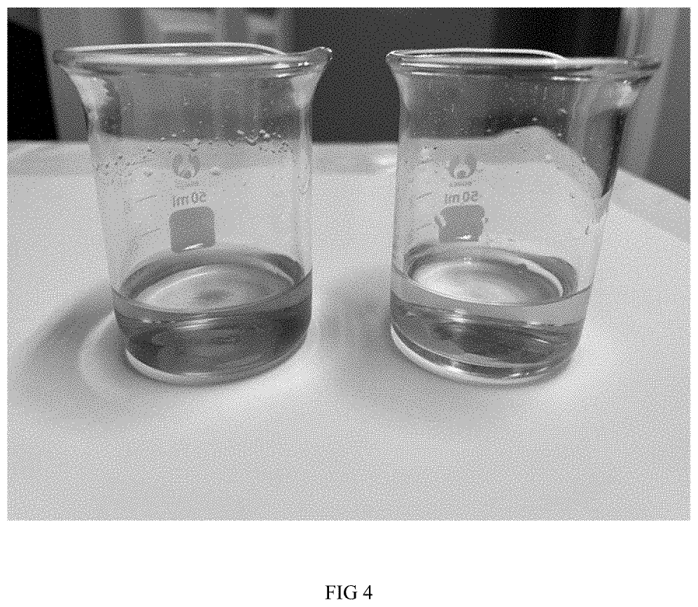

Turning to FIG. 4, an exemplary photo illustrates the Cu+2 complexing ability of glycine. The beaker on the right with the pale blue-green color is a solution with 250 ppm Cu solution as copper gluconate (in 66% ethanol). The beaker on the left is the same solution with 0.12% glycine (that is clear by itself) added which turns to a bright blue color associated with the copper glycine complex indicating the glycine is a much stronger complexing agent than the gluconate and the water in the right beaker. This stronger complexing ability of glycine can be exploited to stabilize these copper salt containing antiseptics by reducing the availability of the Cu+2 ions to precipitate with acids or react with any incompatible compounds in antimicrobial formulation.

Some embodiments include a coordinating or chelating agent that lowers the free copper ion concentration, a preferred agent being glycine, thereby preventing the precipitation of the copper ion with the weak acid buffers.

A result was observed that is an unanticipated benefit attributed to the complexation of the Cu+2 ions in these formulations by the preferred complexation agent glycine. Such formulas were highly stable by inhibiting the formation of dark copper oxides which form when copper containing solutions contact air (oxygen) for a prolonged period of time.

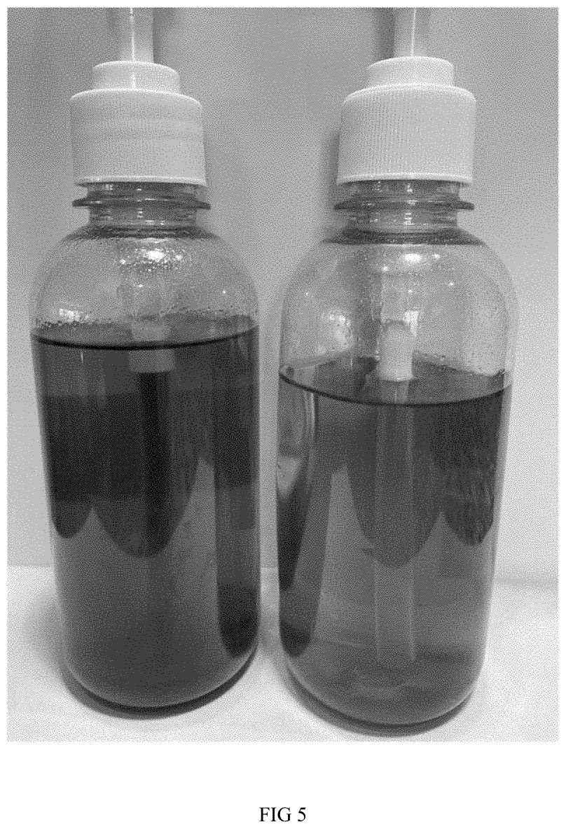

This observation is shown in FIG. 5, which illustrates a comparison of two formulations both stored in similar pump dispenser, clear plastic bottles with the pump mechanism open (as when used by consumers) such that air can reach the liquids. Both formulations were stored these bottles at room temperature for about 14 months. With reference to FIG. 5, the formulation of Reference B (left) is compared the exemplary formulation described in Example 4 (right)). The formulation of Reference B, originally a light green, darkened very noticeably over time, deactivating Cu+2 through the formation of copper oxides. In contrast, the formulation of Example 4 changed very little from the bright blue color of a newly made formula. The formulation of Reference B is an ideal example of the formulations disclosed in U.S. Pat. No. 6,294,186 and is essentially equivalent to the hand sanitizer illustrated Example of U.S. Pat. No. 6,294,186. However, this patent does not reference the instability of their formulation due to exposure to air or the stabilization that might be necessary through the complexation of the Cu+2. This patent does not teach or suggest any reference or Example with the inclusion of glycine or copper glycinate in the patent.

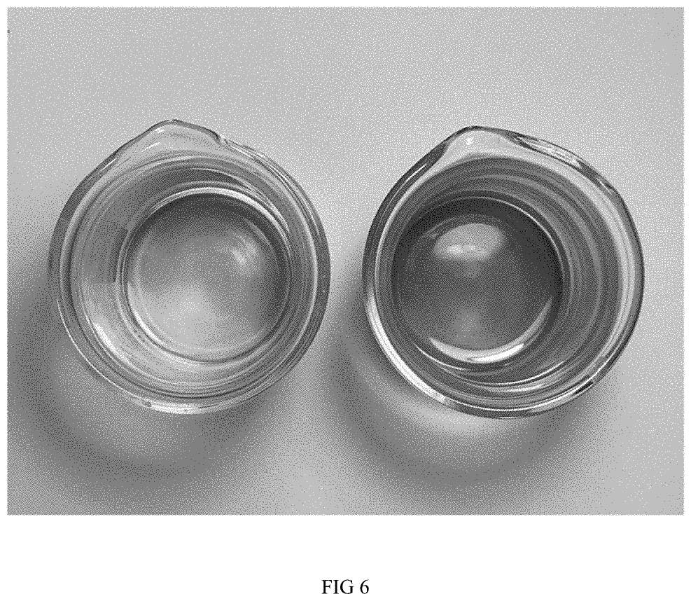

FIG. 6 further clarifies this problem from the instability of antiseptic formulas such as hand sanitizers that contain Cu+2 but are not stabilized with a complexing agent such as glycine. In the photo, the beaker on the left contains 18 ml of a recently prepared sample of Reference B that is light green that can be compared to the beaker on the right with 18 ml of the Reference B sample that was stored in the pump bottle for ˜14 months. The sample on the right contains a dark brown precipitate in suspension that would clearly indicate the formula is unstable and would be very unattractive to consumers. To be clear, when samples of the current invention with glycine are allowed to dry in air for a prolonged period of time, the residue will darken from copper reacting with oxygen. However, this reaction is greatly inhibited in these formulations with complexed copper compared to those without.

The formulation for this Reference B (Table 3) titration comparison is:

TABLE 3

| Ingredient (Wt. %) | Reference B |
|---|---|
| Ethanol (200 proof) | 58.00% |
| Salicylic acid | 2.0% |
| Copper chloride-dihydrate | 0.25% |
| C₁₂-Alkyl dimethyl amine | 0.50% |
| oxide | |
| Hydroxypropyl cellulose | 0.75% |
| (Klucel HF) | |
| Glycerin | 4.0% |
| pH = 2.7 | |
| Water, USP Q.S to 100% | |

The exemplary formulation described in Example 4 (Table 4) for titration comparison is:

TABLE 4

| Ingredient (Wt. %) | Example 4 |
|---|---|
| Ethanol 190 proof) (v/v) | 62.00% |
| Salicylic acid | 0.30% |
| Benzoic acid | 0.20% |
| Sodium salicylate | 1.00% |
| Copper gluconate | 0.18% |
| Glycine | 0.12% |
| Adipic Acid | 0.15% |
| Glacial Acetic Acid | 0.12% |
| Propylene Glycol | 1.00% |
| Glycerin | 4.0% |
| Niacinamide | 0.30% |
| Nicotinic acid | 0.20% |
| DL Panthenol | 0.80% |
| Hydroxypropyl cellulose | 0.50% |
| (Klucel HF) | |
| Sodium lauryl sulfate | 0.50% |
| pH (Adjust to pH 3.7 with 6 N | |
| HCl or 10 N NaOH) | |
| Water, USP Q.S to 100% | |

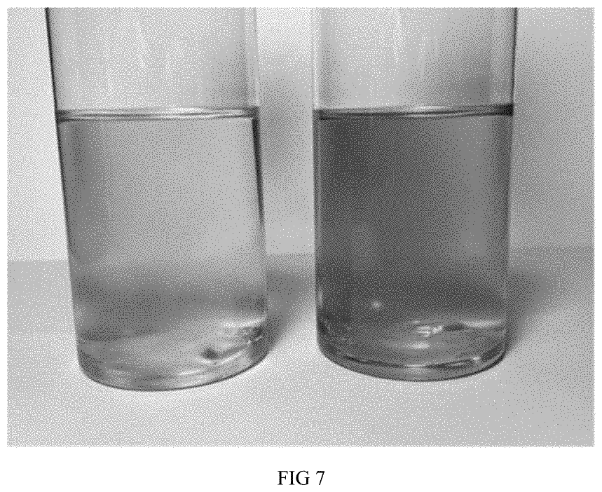

FIG. 7 is a photo that further clarifies the strength of the copper stabilizing characteristic of glycine and the potential to inhibit copper reactions such as the formation of copper oxides. In the PET plastic bottle on the left is ˜40 ml of a hand sanitizer formula that would fall within those described in the U.S. Pat. No. 6,294,186 with the formula of Reference C in table below that contains copper at ˜300 ppm supplied as copper chloride dihydrate. The formula is the light green color characteristic of copper chloride solutions. The bottle on the right is ˜40 ml of the same formula with 0.12% glycine added giving the characteristic blue color of the copper-glycine complex. This demonstrates that in these alcoholic hand sanitizer solutions containing salicylic acid, glycine will provide stronger complexation of the copper than the chloride ions. This stronger complexation of copper is associated with a greater inhibition of coper reactions, in particular, the formation of copper oxides in solution.

The formulation for this Reference C (Table 5) titration comparison is:

TABLE 5

| Ingredient (Wt. %) | Reference C |
|---|---|
| Ethanol 200 proof | 60.00% |
| Salicylic acid | 1.17% |
| Sodium Salicylate | 1.47% |
| Copper chloride dihydate | 0.08% |
| Sodium Lauryl Sulfate | 0.60% |
| Hydroxypropyl cellulose (Klucel | 0.60% |
| HF) | |
| glycerin | 4.00% |
| pH ~3.15 | |
| Water (QS to 100%) | |

Dermatologically Acceptable Carrier

The topical compositions of the present invention also comprise a dermatologically acceptable carrier for the benzoic acid analog and the metal salt. The phrase “dermatologically acceptable carrier”, as used herein, means that the carrier is suitable to come in contact with or for topical application to mammalian keratinous tissue (e.g., human hands, feet, elbows), has good aesthetic properties, is compatible with the actives of the present invention and any other components, and will not cause any untoward safety or toxicity concerns. A safe and effective amount of carrier is from about 50% to about 99. %, preferably from about 80% to about 99.%, more preferably from about 90% to about 98%, and most preferably from about 90% to about 96% of the composition.

The carrier can be in a wide variety of forms. For example, the carrier may be an aqueous-based solution or cleanser, an alcohol-based solution or gel or an emulsion carrier, including, but not limited to, oil-in-water, water-in-oil, water-in-oil-in-water, and oil-in-water-in-silicone emulsions. The carrier solution containing the benzoic acid analog and metal salt can be applied directly to the surface to be treated or delivered via a suitable substrate.

In various embodiments, the carrier may comprise an aqueous solution. Such an aqueous solution may comprise from about 0% to about 98.8%, by weight of the composition, of water.

Alcohol

Additionally, in some embodiments the carrier comprises an alcohol solution. The alcohol may also serve as an active ingredient in the formulation. The amount of alcohol present in the alcohol solution will vary depending on the type of product in which the composition is incorporated into a substrate (such as a wipe) where the amount of alcohol present would be from about 0% to about 40%. For a hand sanitizer, the alcohol solution comprises from about 20% to about 95% of alcohol. Suitable dermatologically acceptable alcohol solutions or gels may comprise from about 0% to about 95%, by weight of the composition, of an alcohol. In some embodiments,

Alcohols suitable for inclusion in the alcohol solutions of the carrier of the present invention include, but are not limited to, monohydric alcohols, dihydric alcohols, and combinations thereof. More preferred alcohols are selected from the group consisting of monohydric linear or branched C2-C18 alcohols. In some embodiments, the alcohol is selected from the group consisting of ethanol, isopropanol, n-propanol, butanol, and combinations thereof. The compositions of the present invention which comprise a carrier comprising an alcohol solution may be anhydrous or water containing.

In some embodiments, the alcohol is in the range of between 25% and 75% by weight of a formulation of an antimicrobial composition. In some embodiments, the alcohol is in the range of between 50% and 70% by weight of a formulation of an antimicrobial composition. In some embodiments, the alcohol is in the range of between 60% and 65% by weight of a formulation of an antimicrobial composition.

Surfactants and Other Antimicrobials:

Usable surface-active agents can include a long list of surfactants used in the personal care and cosmetics industry as these have long safety pedigrees. These include anionic, cationic, non-ionic, and zwitterionic agents. Commonly used surfactant ingredients include: Sodium lauryl sulfate; n-lauroylsarcosine; sodium linear alkylbenzene sulfonate; sodium stearate; 4-(5-dodecyl)benzenesulfonate; docusate (dioctyl sodium sulfosuccinate); alkyl ether phosphates; benzalkaonium chloride (BAC); didecyldimethylammonium chloride; alkyl dimethyl benzyl ammonium saccharinate; cetylpyridinium chloride; perfluorooctanesulfonate (PFOS). Most of these surfactants will add a level of antimicrobial or antiviral activity to the formula. A useful reference for antimicrobial surfactants is Lin, Q. et al, “Sanitizing agents for virus inactivation and disinfection,”

In addition, the water or alcohol-based solutions and gels can include emulsifying surfactants. The emulsifying surfactant comprises from about 0.1% to about 20%, preferably from about 0.1% to 10%, more preferably, from about 0.25% to about 5%, most preferably, from about 0.25% to about 2.5%.

Suitable carriers may also comprise a water containing (i.e., non-alcohol based) emulsion such as oil-in-water emulsions, water-in-oil emulsions, and water-in-silicone emulsions. As will be understood by the skilled artisan, a given component will distribute primarily into either the water or oil/silicone phase, depending on the water solubility/dispersibility of the component in the composition.

Emulsions according to the present invention generally contain a solution as described above and a lipid or oil. Lipids and oils may be derived from animals, plants, or petroleum and may be natural or synthetic (i.e., man-made). Preferred emulsions also contain a humectant, such as glycerin. Emulsions will preferably further contain from about 1% to about 10%, more preferably from about 2% to about 5%, of an emulsifier, based on the weight of the carrier. Emulsifiers may be nonionic, anionic, or cationic.

The emulsion may also contain an anti-foaming agent to minimize foaming upon application to the surface to be treated. Anti-foaming agents include high molecular weight silicones and other materials well known in the art for such use.

In some variations, thickeners can be added to the water or alcohol-based solutions of to form a gel. Examples of suitable thickeners include, but are not limited to, naturally-occurring polymeric materials such as sodium alginate, xanthan gum, quince seed extract, tragacanth gum, starch and the like, semi-synthetic polymeric materials such as cellulose ethers (e.g. hydroxyethyl cellulose, hydroxypropyl cellulose, methyl cellulose, carboxymethyl cellulose, hydroxy propyl methyl cellulose), polyvinylpyrrolidone, polyvinyl alcohol, guar gum, hydroxypropyl guar gum, soluble starch, cationic celluloses, cationic guards and the like and synthetic polymeric materials such as carboxyvinyl polymers, polyvinylpyrrolidone, polyvinyl alcohol, polyacrylic acid polymers, poly methacrylic acid polymers, polyvinyl acetate polymers, polyvinyl chloride polymers, polyvinylidene chloride polymers and the like. Inorganic thickeners may also be used such as aluminum silicates, such as, for example, bentonites, or a mixture of polyethylene glycol and polyethylene glycol stearate or distearate. Also, useful herein are hydrophilic gelling agents. Hydrophobically modified celluloses are also suitable for use in the water or alcohol solutions and gels.

The thickener can be at a concentration of from about 0.01% to about 10%, preferably from about 0.1% to about 5%, and most preferably from about 0.1% to about 3%. Mixtures of the above thickeners may also be used.

Other Ingredients:

It is anticipated that formula compositions may add cosmetically acceptable ingredients known to those skilled in the art of formulation. These would include humectants, emollients, emulsifiers, surfactants, suspending agents, sunscreens, colorants, and fragrances.

For example, lipophilic skin moisturizing agents/emollients may also be incorporated into the water or alcohol-based solutions and gels. Examples of suitable lipophilic skin moisturizers include, but are not limited to, petroleum, mineral oil, micro-crystalline waxes, polyalkenes, paraffin, cerasin, ozokerite, polyethylene, perhydrosqualene, dimethicones, cyclomethicones, alkyl siloxanes, polymethyl siloxanes, methylphenylpolysiloxanes, hydroxylated milk glyceride, castor oil, soy bean oil, maleated soybean oil, safflower oil, cotton seed oil, corn oil, walnut oil, peanut oil, olive oil, cod liver oil, almond oil, avocado oil, palm oil, sesame oil, liquid sucrose octaesters, blends of liquid sucrose octaesters and solid polyol polyesters, lanolin oil, lanolin wax, lanolin alcohol, lanolin fatty acid, isopropyl lanolate, acetylated lanolin, acetylated lanolin alcohols, lanolin alcohol linoleate, lanolin alcohol ricinoleate, beeswax, beeswax derivatives, spermaceti, myristyl myristate, stearyl stearate, carnauba and candelilla waxes, cholesterol, cholesterol fatty acid esters and homologs thereof, lecithin and derivatives, Sphingolipids, ceramides, glycosphingolipids and homologs thereof, and mixtures thereof.

When incorporated into the solutions or gels, the lipophilic skin moisturizer is present at concentrations of from about 0.1% to about 20%, preferably from about 1% to about 15%, more preferably from about 2% to about 10% by weight.

Optionally, the lipophilic skin moisturizing agent can also be thickened using a thickening agent. Suitable thickening agents for the lipophilic skin moisturizing agent include polyacrylates; fumed silica natural and synthetic waxes, alkyl silicone waxes such as behenyl silicone wax; aluminum silicate; lanolin derivatives such as lanosterol; higher fatty alcohols; polyethylene copolymers; areogel; poly ammonium stearate; sucrose esters; hydrophobic clays; petroleum; hydrotalcites; and mixtures thereof.

Various embodiments provide an antimicrobial composition comprising: a safe and effective amount of benzoic acid analog or analogs; a safe and effective amount of a weak organic acid buffer or a combination of weak organic acid buffers with the negative log of the acid dissociation constant, pKa, between about 3.2 and 4.9; and a dermatologically acceptable carrier for the benzoic acid analogs and weak organic acid buffers, wherein the antimicrobial composition has a pH from about 3.2 to 4.9.

In some embodiments, the antimicrobial composition can further comprise a safe and effective amount of an antimicrobial metal ion salt, at a level and pH that does not precipitate with the weak acid buffers. In a preferred embodiment, the antimicrobial metal ion salt is copper gluconate.

In some embodiments, the copper ions are complexed with a coordinating or chelating agent that lowers the free copper ion concentration, thereby preventing the precipitation of the copper ion with the weak acid buffers. In a preferred embodiment, the coordinating or chelating agent is glycine.

Various embodiments provide an antimicrobial formulation comprising: a safe and effective amount of benzoic acid analog or analogs; a safe and effective amount of a weak organic acid buffer or a combination of weak organic acid buffers with the negative log of the acid dissociation constant, pKa, between about 3.2 and 4.9; a safe and effective amount of an antimicrobial copper ion salt; and chelating agent that lowers the free copper ion concentration, thereby preventing the precipitation of the copper ion with the weak acid buffers, wherein the antimicrobial composition has a pH from about 3.4 to 4.6.

Various embodiments provide an antimicrobial formulation comprising: an effective amount of one or more benzoic acid analogs; a combination of weak organic acid buffers having at least 3 pKa, values between 3.2 and 4.9; an effective amount of an antimicrobial copper ion salt; and glycine.

Various embodiments provide an antimicrobial composition comprising: an effective amount of one or more benzoic acid analogs in a weight percent between 0.1% and 20% of the composition; a combination of weak organic acid buffers, having at least 3 different pKa values between 3.5 and 4.9, in a combined weight present between 0.15% and 7% of the composition; an effective amount of an antimicrobial copper ion salt in a weight percent of copper of between 0.0025% and 0.20% of the composition; and glycine in 1.5 to 4 times the molar concentration of copper ions in the composition.

The benzoic acid analog can be selected from the group consisting of benzoic acid, salicylic acid, and combinations thereof. The chelating agent can be glycine.

The antimicrobial copper ion salt can be copper gluconate. However, the copper ion salt can be selected from the group consisting of copper sulfate, copper chloride, copper nitrate, copper acetate, copper bromide, copper iodide, copper glycinate, and combinations thereof. The benzoic acid analog can be selected from the group consisting of benzoic acid, salicylic acid, and combinations thereof.

In some embodiments, the buffering system of multiple weak acids in low concentrations can be a combination of adipic acid (pKa=4.43), gluconic acid (pKa=3.85), and acetic acid (pKa=4.76). In some embodiments, buffering system of multiple weak acids in low concentrations can be a combination of three or more of: adipic acid (pKa=4.43), nicotinic acid (pKa=4.82), glutaric acid (pKa=3.76), gluconic acid (pKa=3.85), lactic acid (pKa=3.86), and acetic acid (pKa=4.76). In some examples, the buffering system of multiple weak acids in low concentrations can be a combination of three or more of: adipic acid (pKa=4.43), nicotinic acid (pKa=4.82), glutaric acid (pKa=3.76), gluconic acid (pKa=3.85), lactic acid (pKa=3.86), and acetic acid (pKa=4.76). includes a buffering system of multiple weak acids in low concentrations: lactic acid (pKa=3.86, 0.26%), adipic acid (pKa=4.43, 0.15%), and glacial acetic acid (pKa=4.76, 0.12%).

Some embodiments provide a hand sanitizer solution comprising the antimicrobial composition. The hand sanitizer solution can further comprise a dermatologically acceptable carrier for the antimicrobial composition.

The dermatologically acceptable carrier can comprise an alcohol solution. The alcohol solution can comprise monohydric alcohols, dihydric alcohols, and combinations thereof. Suitable dermatologically acceptable alcohol solutions or gels may comprise from about 0% to about 95%, by weight of the hand sanitizer composition.

The alcohol solution can comprise a surfactant. The surfactant can be selected from the group consisting of anionic surfactants, cationic surfactants, amphoteric surfactants, and combinations thereof. The surfactant can comprise from about 0.25% to about 5% by weight of the hand sanitizer composition.

The dermatologically acceptable carrier can comprise an aqueous solution may comprise from about 0% to about 98%, by weight of the composition, of water.

The aqueous solution can comprise a surfactant. The surfactant can be selected from the group consisting of anionic surfactants, cationic surfactants, amphoteric surfactants, and combinations thereof. The surfactant can comprise from about 0.25% to about 5% by weight of the hand sanitizer composition.

A method of reducing absenteeism of persons from school and/or work wherein said absenteeism is caused by bacterial or viral illness, said method comprising; topically applying the antimicrobial formulation to a bodily surface of a person which is prone to viral and/or bacterial contamination; rubbing said surface for at least 15 seconds; and allowing said surface to dry. The bodily surface can be one or more hands.

Various embodiments provide an antimicrobial product comprising the antimicrobial formulation. The antimicrobial product can be a wipe product suitable for personal care use. The antimicrobial product can be a hand sanitizer lotion, gel, or spray suitable for personal care use.

The antimicrobial product can be a personal care product. For example, the personal care product can be selected from the group consisting of hand soaps, hand sanitizers, foot sanitizers, skin antiseptics, body washes, shower gels, body lotions, and combinations thereof.

The antimicrobial product can be a household care product. For example, the household care product can be selected from the group consisting of hard surface cleaners, dish detergents, and floor waxes.

Various embodiments provide an antimicrobial active ingredient formulation comprising: an effective amount of one or more benzoic acid analogs in a weight percent between 60% and 65% of the formulation; a combination of weak organic acid buffers, having at least 3 different pKa values between 3.5 and 4.9, in a weight present between 15% and 30% of the formulation; an effective amount of an antimicrobial copper ion salt in a weight percent of between 5% and 15% of the formulation; and glycine in a weight percent of between 5% and 10% of the formulation.

An antimicrobial composition can comprise: the antimicrobial active ingredient formulation; a dermatologically acceptable carrier; and water. the dermatologically acceptable carrier can comprise from about 75% to about 98% weight percent of the antimicrobial composition. The antimicrobial formulation can comprise from about 0.25% to about 25% by weight of the antimicrobial composition.

Some embodiments provide a hand sanitizer composition comprising the antimicrobial active ingredient formulation. The hand sanitizer composition can further comprise a dermatologically acceptable carrier for the antimicrobial active ingredient formulation. The antimicrobial active ingredient formulation can comprise from about 0.25% to about 25% by weight of the hand sanitizer composition.

Some embodiments include methods of making the antimicrobial formulations. A method of producing an exemplary antimicrobial formulation can include the following steps: Start with water; add the copper salt to the water; add the copper complexing agent to the copper in solution; the addition of the copper complexing agent makes the copper salt soluble in the aqueous solution. The next steps include adding an alcohol to the aqueous solution then adding one or more benzoic acid analogs, typically salicylic acid or a salt of a salicylic acid to the solution. The buffering system of multiple weak acids each having a different pKa value between 3.2 and 4.9, and each in a low concentration is added to the solution. After mixing the buffering system of multiple weak acids, other components, as described herein may be added to the solution. Adding can also mean mixing into and can include changing of temperature either hotter or colder.

In some embodiments, a method of producing an exemplary antimicrobial formulation can include the following steps: Start with water; then mixing copper gluconate to the water; then mixing in glycine to the water in a ratio between 1:1 and 4:1 mixing copper gluconate. The glycine complexes with the copper in the water and the resulting copper complex is water soluble. The aqueous solution comprising the copper complex contains no copper precipitates.

The next steps include adding EtOH to the aqueous solution then mixing in one or more benzoic acid analogs, typically salicylic acid or a salt of a salicylic acid to the solution. In some examples, the EtOH is in a concentration of between 60 and 70 weight percent of the total antimicrobial formulation. In some examples, the total of the one or more benzoic acid analogs is in a concentration of between 0.5 and 1.5 weight percent of the total antimicrobial formulation. In some examples, the total of the one or more benzoic acid analogs is benzoic acid in a concentration of between 0.1 and 0.4 weight percent of the total antimicrobial formulation, salicylic acid in a concentration of between 0.2 and 0.5 weight percent of the total antimicrobial formulation, and a salt of a salicylic acid in a concentration of between 0.8 and 1.2 weight percent of the total antimicrobial formulation.

The buffering system of multiple weak acids each having a different pKa value between 3.2 and 4.9, and each in a low concentration is added to the solution. After mixing the buffering system of multiple weak acids, other components, as described herein may be added to the solution. In some examples, the buffering system of multiple weak acids in low concentrations can be a combination of adipic acid (pKa=4.43), gluconic acid (pKa=3.85), and acetic acid (pKa=4.76). In some embodiments, the buffering system of multiple weak acids in low concentrations can be a combination of adipic acid (pKa=4.43), gluconic acid (pKa=3.85), and acetic acid (pKa=4.76). In some embodiments, buffering system of multiple weak acids in low concentrations can be a combination of three or more of: adipic acid (pKa=4.43), nicotinic acid (pKa=4.82), glutaric acid (pKa=3.76), gluconic acid (pKa=3.85), lactic acid (pKa=3.86), and acetic acid (pKa=4.76). In some examples, the buffering system of multiple weak acids in low concentrations can be a combination of three or more of: adipic acid (pKa=4.43), nicotinic acid (pKa=4.82), glutaric acid (pKa=3.76), gluconic acid (pKa=3.85), lactic acid (pKa=3.86), and acetic acid (pKa=4.76). includes a buffering system of multiple weak acids in low concentrations: lactic acid (pKa=3.86, 0.26%), adipic acid (pKa=4.43, 0.15%), and glacial acetic acid (pKa=4.76, 0.12%).

Any of the surfactants, other antimicrobials, vitamins, humectants, emollients, emulsifiers, surfactants, suspending agents, sunscreens, colorants, waxes, fragrances, and lipophilic skin moisturizing agents, described herein, may be mixed into the antimicrobial formulation, as desired. As described in the Examples, the pH of the antimicrobial formulation can be adjusted to between 3.0 and 4.5 with HCl or NaOH.

### EXAMPLES

Various non-limiting examples are described below.

Various embodiments provide an antimicrobial composition for a hand sanitizer solution, the composition comprising: an effective amount of one or more benzoic acid analogs in a weight percent between 0.1% and 20% of the formulation; a combination of weak organic acid buffers, having at least 3 different pKa values between 3.2 and 4.9, in a combined weight present between 0.15% and 7% of the formulation; an effective amount of an antimicrobial copper ion salt in a weight percent of copper of between 0.0025% and 0.20% of the formulation; and glycine in 1.5 to 4 times the molar concentration of copper ions in the formulation.

In some embodiments, the one or more benzoic acid analogs can be selected from the group consisting of salicylic acid, benzoic acid, salts thereof, and combinations thereof. In some embodiments, the antimicrobial copper ion salt can be copper gluconate. In some embodiments, the buffering system of multiple weak acids in low concentrations can be a combination of adipic acid (pKa=4.43), gluconic acid (pKa=3.85), and acetic acid (pKa=4.76).

Accordingly, the hand sanitizer composition can comprise: the antimicrobial formulation and a dermatologically acceptable carrier. For example, the hand sanitizer composition can comprise: the antimicrobial formulation in a weight percent between 1% and 5% of the composition, the dermatologically acceptable carrier in a weight percent between 60% and 95% of the composition, and water. The dermatologically acceptable carrier can comprise one or more alcohols in a weight percent of at least 50% of the composition. In addition, the dermatologically acceptable carrier can comprise at least of a surfactant, an emulsifier, and a humectant. The hand sanitizer composition can be formulated as a gel, a lotion, or a spray.

### Exemplary Antimicrobial Formulations

### Example 1

Table 6 illustrates an exemplary formulation of a hand sanitizer.

TABLE 6

| Ingredient (Wt. %) | Example 1 |
|---|---|
| Ethanol (SD alcohol 40-B, 200 | 70.00% |
| proof) (v/v) | |
| Benzoic acid | 0.65% |
| Sodium salicylate | 0.85% |
| Nicotinic Acid | 0.50% |
| Adipic Acid | 0.60% |
| Acetic acid (1 N aqueous) | 2.50% |
| DL Panthanol | 1.00% |
| Glycerin | 5.00% |
| Propylene Glycol | 1.00% |
| Vitamin E acetate | 0.30% |
| Acrylic acid polymer, | 0.20% |
| Carbomer; Carbopol Ultrez 30 | |
| Polysorbate 80 | 1.70% |
| ammonium lauryl sulfate | 0.60% |
| pH (Adjust to pH 4.2 with 6 N | |
| HCl or 10 N NaOH) | |
| Water, USP Q.S to 100% | |

### Example 2

Table 7 illustrates an exemplary formulation of a hand sanitizer.

TABLE 7

| Ingredient (Wt. %) | Example 2 |
|---|---|
| Ethanol (SD alcohol 40-B, 200 | 65.00% |
| proof) (v/v) | |
| Salicylic acid | 0.34% |
| Benzoic acid | 0.20% |
| Sodium salicylate | 1.00% |
| Copper sulfate pentahydrate | 0.10% |
| Glycine | 0.12% |
| Nicotinic acid | 0.20% |
| Adipic Acid | 0.15% |
| Acetic acid (1 N aqueous) | 3.50% |
| Propylene Glycol | 1.00% |
| Glycerin | 6.00% |
| DL Panthenol | 0.50% |
| Hydroxypropyl cellulose | 0.50% |
| (Klucel HF) | |
| Polysorbate 80 | 0.50% |
| Dodecyldimethyl-amine oxide | 1.00% |
| pH (Adjust to pH 3.6 with 6 N | |
| HCl or 10 N NaOH) | |
| Water, USP Q.S to 100% | |

### Example 3

Table 8 illustrates an exemplary formulation of a hand sanitizer.

TABLE 8

| Ingredient (Wt. %) | Example 3 |
|---|---|
| Ethanol (SD alcohol 40-B, 200 | 60.00% |
| proof) (v/v) | |
| Salicylic acid | 0.35% |
| Benzoic acid | 0.20% |
| Sodium salicylate | 1.00% |
| Copper gluconate | 0.22% |
| Glycine | 0.15% |
| Lactic acid | 0.26% |
| Adipic Acid | 0.15% |
| Glacial Acetic Acid | 0.12% |
| Propylene Glycol | 1.00% |
| Glycerin | 3.60% |
| Niacinamide | 0.50% |
| DL Panthenol | 0.80% |
| Hydroxypropyl cellulose | 0.55% |
| (Klucel HF) | |
| Sodium lauryl sulfate | 0.60% |
| pH (Adjust to pH 3.6 with 6 N | |
| HCl or 10 N NaOH) | |
| Water, USP Q.S to 100% | |

### Example 4

Table 9 illustrates an exemplary formulation of a hand sanitizer.

TABLE 9

| Ingredient (Wt. %) | Example 4 |
|---|---|
| Ethanol 190 proof) (v/v) | 62.00% |
| Salicylic acid | 0.30% |
| Benzoic acid | 0.20% |
| Sodium salicylate | 1.00% |
| Copper gluconate | 0.18% |
| Glycine | 0.12% |
| Adipic Acid | 0.15% |
| Glacial Acetic Acid | 0.12% |
| Propylene Glycol | 1.00% |
| Glycerin | 4.0% |
| Niacinamide | 0.30% |
| Nicotinic acid | 0.20% |
| DL Panthenol | 0.80% |
| Hydroxypropyl cellulose | 0.50% |
| (Klucel HF) | |
| Sodium lauryl sulfate | 0.50% |
| pH (Adjust to pH 3.7 with 6 N | |
| HCl or 10 N NaOH) | |
| Water, USP Q.S to 100% | |

### Example 5

Table 10 illustrates an exemplary formulation of a hand sanitizer.

TABLE 10

| Ingredient (Wt. %) | Example 5 |
|---|---|
| Ethanol (SD alcohol 40-B, 200 | 20.00% |
| proof) (v/v) | |
| Salicylic acid | 0.25% |
| Benzoic acid | 0.15% |
| Sodium salicylate | 1.10% |
| Copper gluconate | 0.18% |
| Glycine | 0.12% |
| Adipic Acid | 0.20% |
| Acetic acid (1 N aqueous) | 2.50% |
| Nicotinic acid | 0.20% |
| Propylene Glycol | 1.50% |
| Glycerin | 8.00% |
| Niacinamide | 0.30% |
| DL Panthenol | 0.60% |
| Acrylic acid polymer, | 0.10% |
| Carbomer; Carbopol Ultrez 30 | |
| Polysorbate 80 | 0.50% |
| ammonium lauryl sulfate | 0.80% |
| Fragrance | 0.05% |
| pH (Adjust to pH 3.8 with 6 N | |
| HCl or 10 N NaOH) | |
| Water, USP Q.S to 100% | |

Hand Sanitizer Wipes

Various embodiments provide an antimicrobial formulation for a hand wipe solution, the formulation comprising: an effective amount of one or more benzoic acid analogs in a weight percent between 0.1% and 20% of the formulation; a combination of weak organic acid buffers, having at least 3 different pKa values between 3.2 and 4.9, in a combined weight present in a range between 0.15% and 7% of the formulation; an effective amount of an antimicrobial copper ion salt in a weight percent of copper in a range between 0.0025% and 0.20% of the formulation; and glycine in 1.5 to 4 times the molar concentration of copper ions in the formulation.

In some embodiments, the one or more benzoic acid analogs can be selected from the group consisting of salicylic acid, benzoic acid, salts thereof, and combinations thereof. In some embodiments, the antimicrobial copper ion salt can be copper gluconate. In some embodiments, the buffering system of multiple weak acids in low concentrations can be a combination of three or more of: adipic acid (pKa=4.43), nicotinic acid (pKa=4.82), glutaric acid (pKa=3.76), gluconic acid (pKa=3.85), lactic acid (pKa=3.86), and acetic acid (pKa=4.76).

Various embodiments provide an antimicrobial active ingredient formulation comprising: an effective amount of one or more benzoic acid analogs in a weight percent between 55% and 65% of the formulation; a buffering system of multiple weak acids in low concentrations, having at least 3 different pKa values between 3.6 and 4.9, in a weight present between 20%-25% of the formulation; an effective amount of an antimicrobial copper ion salt in a weight percent of between 10%-12% of the formulation; and glycine in a weight percent of between 7%-8% of the formulation.

Some embodiments provide a hand wipe solution comprising the antimicrobial active ingredient formulation. Accordingly, the hand wipe solution can comprise: the antimicrobial active ingredient formulation and a dermatologically acceptable carrier. For example, the hand wipe solution can comprise: the antimicrobial active ingredient formulation in a weight percent between 1% and 5% of the wipe solution, the dermatologically acceptable carrier in a weight percent between 60% and 95% of the wipe solution, and water. The dermatologically acceptable carrier can comprise one or more alcohols in a weight percent of at least 50% of the wipe solution. In addition, the dermatologically acceptable carrier can comprise at least of a surfactant, an emulsifier, and a humectant.

Wipe solution may be applied to chosen wipe substrate (natural or synthetic, woven, or non-woven) at a loading of 0.5 to 3.0 g lotion/g substrate by pouring lotion onto one or multiple wipes (contained inside plastic bag, pouch, or container) and applying/releasing pressure until lotion is evenly wicked into the substrate materials. For each of the examples above, apply the wipe product to a person's skin (e.g., hands) as needed, though recommended three to five times daily, to provide immediate and residual anti-viral and antibacterial efficacy to inhibit the transmission of bacterial or viral diseases.

Additional carriers suitable for the wipe solution of the present invention may include various substrate-based products. In such instances, the present compositions may be impregnated into or onto the substrate products and may be allowed to remain wet or may be subjected to a drying process. For instance, suitable carriers include, but are not limited to, dry and wet wipes suitable for personal care and household use (e.g., nonwoven baby wipes, household cleaning wipes, surgical preparation wipes, etc.); diapers; infant changing pads; dental floss; personal care and household care sponges or woven cloths (e.g., washcloths, towels, etc.); tissue-type products (e.g. facial tissue, paper towels, etc.); and disposable garments (e.g., gloves, smocks, surgical masks, infant bibs, socks, shoe inserts, etc.).

### Examples of Wipe Solutions

### Example 6

Table 11 illustrates an exemplary formulation infused into sanitizer skin wipes.

TABLE 11

| Ingredient (Wt. %) | Example 6 |
|---|---|
| Benzoic acid | 0.30% |
| Salicylic acid | 0.40% |
| Sodium Salicylate | 2.00% |
| Dimethicone (skin feel) | 0.75% |
| Propylene glycol | 0.80% |
| Adipic acid | 0.60% |
| Acetic acid (1 N aqueous) | 1.50% |
| Palmitic acid | 0.20% |
| DL Panthenol | 1.00% |
| Nicotinic acid | 0.50% |
| Fragrance | 0.05% |
| Ethanol (SD alcohol 40-B, | 12.00% |
| 200 proof) (v/v) | |
| pH (Adjust to pH 3.9 with 6 N | |
| HCl or 10 N NaOH) | |
| Water to QS 100% | |

### Example 7

Table 12 illustrates an exemplary formulation infused into sanitizer skin wipes.

TABLE 12

| Ingredient (Wt. %) | Example 7 |
|---|---|
| Salicylic acid | 0.40% |
| Sodium Salicylate | 1.50% |
| Copper gluconate | 0.36% |
| Glycine | 0.24% |
| Dimethicone (skin feel) | 0.50% |
| Propylene glycol | 1.00% |
| Dodecyldimethyl-amine oxide | 1.00% |
| Lactic acid | 0.35% |
| Glutaric acid | 0.15% |
| Acetic acid (glacial) | 0.15% |
| Tocopheryl acetate | 0.30% |
| Nicotinic acid | 0.25% |
| DL Panthenol | 0.40% |
| Fragrance | 0.05% |
| Ethanol (SD alcohol 40-B, | 10.00% |
| 200 proof) (v/v) | |
| pH (Adjust to pH 3.6 with 6 N | |
| HCl or 10 N NaOH) | |
| Water to QS 100% | |

As used herein, the phrase “at least one of A, B, and C” can be construed to mean a logical (A or B or C), using a non-exclusive logical “or;” however, it can be construed to mean (A, B, and C); in addition, it can be construed to mean (A and B) or (A and C) or (B and C). As used herein, the phrase “A, B and/or C” should be construed to mean (A, B, and C) or alternatively (A or B or C), using a non-exclusive logical “or.”

The present invention has been described above with reference to various exemplary embodiments and examples, which are not intended to be limiting in describing the full scope of systems and methods of this invention. However, those skilled in the art will recognize that equivalent changes, modifications and variations of the embodiments, materials, systems, and methods may be made within the scope of the present invention with substantially similar results and are intended to be included within the scope of the present invention, as set forth in the following claims.

1. An antimicrobial composition comprising:
a) a safe and effective amount of benzoic acid analog or analogs;
b) a safe and effective amount of a weak organic acid buffer or combination of buffers with the negative log of the acid dissociation constant, pKa, between about 3.2 and 4.9; and
c) a dermatologically acceptable carrier for the benzoic acid analogs and acid buffers
wherein the antimicrobial composition has a pH from about 3.2 to 4.9.

2. The antimicrobial composition of claim 1, further comprising a safe and effective amount of an antimicrobial metal ion salt, at a level and pH that does not precipitate with the weak acid buffers.

3. The antimicrobial composition of claim 2, wherein the antimicrobial metal ion salt is copper gluconate.

4. The antimicrobial composition of claim 2, further comprising chelating agent that lowers the free copper ion concentration by complexing to a copper ion, preventing the precipitation of the copper ion with the weak acid buffers.

5. The antimicrobial composition of claim 2, further comprising chelating agent that lowers the free copper ion concentration by complexing to a copper ion, inhibiting the precipitation of copper oxide on exposure to air.

6. The antimicrobial composition of claim 4, wherein the chelating agent is glycine.

7. The antimicrobial composition of claim 5, wherein the chelating agent is glycine.

8. An antimicrobial formulation comprising:
an effective amount of one or more benzoic acid analogs;
a combination of weak organic acid buffers having at least 3 pKa, values between 3.2 and 4.9;
an effective amount of an antimicrobial copper salt;
a chelating agent;
a dermatologically acceptable carrier; and
water.

9. The formulation of claim 8 wherein the one or more benzoic acid analogs is selected from the group consisting of benzoic acid, salicylic acid, and combinations thereof.

10. The formulation of claim 8 wherein said copper salt is selected from the group consisting of copper gluconate, copper glycinate, copper sulfate, copper chloride, copper salicylate, copper nitrate, copper acetate, copper bromide, copper iodide, and combinations thereof.

11. The formulation of claim 8, wherein the dermatologically acceptable carrier comprises from about 75% to about 98% weight percent of the formulation.

12. The formulation of claim 8, wherein said carrier comprises an alcohol solution.

13. The formulation of claim 12 wherein said alcohol solution comprises monohydric alcohols, dihydric alcohols, and combinations thereof.

14. The formulation of claim 8, wherein said composition further comprises a surfactant.

15. The formulation of claim 14 wherein said surfactant is selected from the group consisting of anionic surfactants, cationic surfactants, amphoteric surfactants, and combinations thereof.

16. The formulation of claim 8, wherein:
the one or more benzoic acid analogs is in a weight percent between 0.1% and 20% of the formulation;
the combination of weak organic acid buffers is in a combined weight present between 0.15%-7% of the formulation;
an effective amount of an antimicrobial copper ion salt is in a weight percent of copper of between 0.0025%-0.20% of the formulation; and
the glycine is in 1.5 to 4 times the molar concentration of copper ions in the formulation.

17. An antimicrobial product comprising said formulation of claim 8.

18. The product of claim 17 wherein said product is a personal care product.

19. The product of claim 17 wherein said personal care product is selected from the group consisting of hand soaps, hand sanitizers, body washes, shower gels, body lotions, and combinations thereof.

20. The product of claim 17 wherein said product is a household care product.

21. The product of claim 20 wherein said product is a household care product selected from the group consisting of hard surface cleaners, dish detergents, filter cleaners, and floor waxes.

22. The product of claim 17 wherein said product is a wipe product suitable for personal care use.

23. An antimicrobial composition comprising:
an antimicrobial active formulation consisting of:
an effective amount of one or more benzoic acid analogs in a weight percent between 61% and 63% of the formulation;
a combination of weak organic acid buffers, having at least 3 different pKa values between 3.2 and 4.9, in a weight present between 24%-27% of the formulation;
an effective amount of an antimicrobial copper ion salt in a weight percent of between 7%-8% of the formulation; and
glycine in a weight percent of about 5% of the formulation;
a dermatologically acceptable carrier; and
water.

24. The antimicrobial composition of claim 23, wherein the combination of weak acid buffers is a combination of adipic acid (pKa=4.43), nicotinic acid (pKa=4.82), and acetic acid (pKa=3.75).

25. The antimicrobial composition of claim 23, wherein the combination of weak acid buffers is a combination of three or more of: adipic acid (pKa=4.43), nicotinic acid (pKa=4.82), glutaric acid (pKa=3.76), gluconic acid (pKa=3.85), lactic acid (pKa=3.86), and acetic acid (pKa=4.76).

26. The antimicrobial composition of claim 23, wherein the antimicrobial active formulation comprises from about 0.25% to about 25% by weight of the antimicrobial composition.

27. The antimicrobial composition of claim 26, wherein the dermatologically acceptable carrier comprises from about 75% to about 98% weight percent of the antimicrobial composition.
